(19) United States
(12) Patent Application Publication
(10) Pub. No.: US 2025/0187913 A1
(43) Pub. Date: Jun. 12, 2025

(54) METHOD FOR REMOVING AND IMMOBILISING CARBON DIOXIDE FROM THE ATMOSPHERE AND/OR A WASTE GAS

(71) Applicant: FLD TECHNOLOGIES GMBH, Offenbach am Main (DE)

(72) Inventor: Marek FULDE, Offenbach (DE)

(21) Appl. No.: 18/841,133

(22) PCT Filed: Feb. 24, 2023
(86) PCT No.: PCT/EP2023/054656

§ 371 Date: Aug. 23, 2024

(30) Foreign Application Priority Data
Feb. 25, 2022 (EP) 22158779.3

(51) Int. Cl. C01B 3/24; C01B 32/05; C10G 2/00; C12P 5/02

(52) U.S. Cl. CPC C01B 3/24; C01B 32/05; C10G 2/50; C12P 5/023; C01B 2203/0272; C01B 2203/04; C01B 2203/062; C01B 2203/148; C01B 2203/84; C01B 2203/86

(57) ABSTRACT

The invention relates to a method for removing and immobilising carbon dioxide (102) from the atmosphere and/or a waste gas (113), comprising the steps of: a) converting the carbon dioxide (102) from the atmosphere and/or a waste gas (113) into biomass (100) by way of photosynthesis, preferably on agricultural surfaces, in particular in a greenhouse (1); b) carrying out a biogas reaction, in which the biomass (100) produced in step a) is converted into biogas (101), containing methane (103) and carbon dioxide (102), in particular in a biogas reactor (2); c) separating the methane (103) from the obtained biogas (101); d) splitting the methane (103) into carbon (106) and hydrogen (110), the carbon (106) being obtained as a solid; e) collecting the obtained carbon (106); f) depositing the obtained carbon (106); and g) reacting carbon dioxide (102) from the biogas with the hydrogen (110) obtained in step d) to form a hydrocarbon mixture.

The invention relates to a process for removing and immobilizing carbon dioxide (CO₂) from the atmosphere and/or from an offgas. This involves taking up CO₂ from the air and/or an offgas and converting it into solid carbon which is stored for a long period of time.

What are known as “carbon dioxide capture and storage” (CCS) processes are known to those skilled in the art for the reduction of CO₂ in the atmosphere. The greenhouse effect has been known for around 200 years (J. B. Fourier), and it was discussed at the First World Climate Conference in Geneva in 1979. The removal from the atmosphere and immobilization of greenhouse gases, especially CO₂, is also referred to as sequestration and comprises the capture of CO₂ from industrial sources and in power plants, transport to a storage site and ultimately long-term storage of CO₂, with the latter being isolated from the atmosphere.

In order to avoid CO₂ emissions, known CCS processes capture CO₂ and then store the gas. When using this process, the offgas to be treated is first freed of particles and sulfur compounds. In the following step, CO₂ is separated off from the offgas, compressed, optionally transported away and stored underground. It is disputed whether lasting binding of CO₂ with minerals takes place here or whether release of the gas back into the atmosphere should be anticipated. In addition, there is the risk that subterranean injection of CO₂ under high pressure could lead to earthquakes. Moreover, mixing with and dissolution of CO₂ in groundwater could cause what are known as “cold-water geysers”, which likewise would cause re-emission.

A further drawback of CCS technology resides in the lack of infrastructure for the necessary transport of CO₂. Emission sources such as power plants or cement plants are usually remote from suitable CO₂ storage sites, which would require the construction of suitable pipelines.

DE 10 2013 112 205 A1, in contrast, suggests that carbon in solid form can for example be stored in old coal seams over the long term and without any problems.

Kamman et al., in “Biokohle: Ein Weg zur dauerhaften Kohlenstoff-Sequestrierung” [Biochar: A route to long-term carbon sequestration], Environmental Monitoring and Climate Impact Research Station Linden, Hessian Agency for the Environment and Geology, Department of Plant Ecology of the Justus Liebig University Gießen, April 2010, pages 1 to 8, DOI 14902790298116661323, discuss the use of biochar in soils.

DE 10 2007 037 672 A1 describes a process for harmonizing electricity supply/consumption patterns by means of intermediate storage and incorporation of CO₂ recovery. Pure CO₂ is obtained from CO₂-containing offgas and CO₂ is subjected to intermediate storage or final storage, which corresponds to a known CCS process. The cracking of hydrocarbons is not envisaged and there is no storage of carbon in solid form.

WO 2015/044407 A1 relates to a process for storing electricity from renewable sources. This process involves reacting pure hydrogen, obtained by electrolysis of water, with pure CO₂ or a CO₂/CO/H₂ mixture to give methane. Methane is intermediately stored and then cracked to give carbon and hydrogen. The carbon is reused for the production of CO₂ or a CO₂/CO/H₂ mixture. CO₂ is not stored or removed from the atmosphere because the carbon is passed through a complete cycle in the process.

J. Temple in “Klima-Ingenieure machen Ernst, CO₂ aus der Luft waschen” [The gloves are off: Climate engineers look to scrubbing CO₂ from the skies], Technology Review, pages 49 to 52, June 2017, describes the scrubbing of CO₂ from the air for plant fertilization. For the separation, adsorbent-coated filters are used, which must be heated for the desorption of CO₂, with corresponding energy requirements.

WO 2014/170184 A1 relates to an adsorption apparatus for the separation of gas, in particular for CO₂, as does WO 2015/185434 A1.

WO 2016/005226 A1 is directed to a process for cyclic adsorption/desorption in CCS processes.

Wurzbacher, J. A., in “Development of a temperature-vacuum swing process for CO₂ capture from ambient 2015, air”, ETH Zurich Research Collection, https://doi.org/10.3929/ethz-a-010432423, describes a process for extraction of concentrated CO₂ from the atmosphere by means of adsorption.

Gebald, C., in “Development of amine-functionalized adsorbent for carbon dioxide capture from atmospheric air”, 2014, ETH Zürich Research Collection, https://doi.org/10.3929/ethz-a-010171623, discloses the use of aminosilanes as adsorbents which are used for binding CO₂ from the atmosphere.

In addition, documents U.S. Pat. No. 9,095,813, US 2015/14815661, U.S. Pat. Nos. 8,119,091, 8,728,428, 9,975,100, 8,871,008, 9,637,393, US 2017/15622883, US 2017/15591324, and CA 2017051581 are directed to the removal of CO₂ from the atmosphere.

E. Katifu, in “Carbon dioxide absorption using fresh water algae and identifying potential uses of algal biomass”, Faculty of Engineering and the Built Environment, University of the Witwatersrand, 2011, Johannesburg, deals with the use of microalgae for the reduction of CO₂ emissions.

The issue of the removal of emitted CO₂ from the air (Carbon Dioxide Removal, CDR) is becoming increasingly important. It is now considered certain that the climate goal of limiting the temperature rise to 1.5° C., or 2.0° C., cannot be achieved by avoiding emissions alone. Instead, continuous CO₂ removal from the atmosphere appears to be necessary, which by the year 2100 should comprise 100 GT to 1000 GT of CO₂ according to the IPCC Special Report “Global warming of 1.5° C.” of the Intergovernmental Panel on Climate Change (IPCC), October 2018.

Only a few processes exist for capturing already-released CO₂ from the atmosphere.

These can essentially be divided into four groups:
i) combustion of biomass and storage of CO₂: BECCS (bioenergy with carbon capture and storage),
ii) removal of CO₂ from the ambient air by physical and/or chemical sorption processes, in particular by chemisorption using amines, and subsequent injection-DACCS (direct air carbon capture and storage),
iii) enrichment of carbon in fields by adding biochar,
iv) processes which increase the ability of the soil or water to absorb CO₂. For instance, crushed minerals are spread over areas of land and assist the chemical binding of CO₂ from the air. Furthermore, ocean alkalinization has been proposed, whereby ground minerals lower the pH of the water and thus promote the storage of CO₂ in the sea.

The BECCS and DACCS processes are based on capturing CO₂ and sequestering it underground. In the BECCS process, biomass is used as fuel and biogenic CO₂ is removed from the offgas. In the DACCS process, CO₂ is extracted from the ambient air by means of sorption processes, i.e. chemical or physical processes. The gaseous CO₂ must then be transported to spatially remote storage facilities and stored geologically.

In the BECCS and DACCS processes, and CCS processes in general, CO₂ is separated and stored as such, this having known disadvantages because of the injection and storage of gases.

In the DACCS process, ambient air is conveyed by means of fans through adsorption material, with CO₂ being physically and/or chemically bound at the surface of the adsorption material. After a saturation equilibrium is reached, CO₂ is desorbed in a second step by heating so that the adsorption material is regenerated. Typically, such systems employ at least two apparatuses that operate with a phase offset, i.e. in alternating fashion, with adsorption taking place in one of the apparatuses while regeneration is conducted in the second apparatus. Such an alternation is also referred to as a swing process. It is a feature of this process that considerable amounts of air have to be filtered, which is due to the low concentration of CO₂ in the air (approx. 400 ppm).

Gaseous CO₂ is produced, which for example must be stored geologically for the long term. Storage in gas form is thus necessary here too.

With a view to the CO₂ balance, the operation of a DACCS plant also requires the availability of emissions-free energy for the generation of the heat that is needed for the desorption, and also the suitability of geological formations for the storage of the gaseous CO₂.

Among the further possibilities for binding CO₂, the deposition of biomass in green form, for example by compositing, is not constructive for ensuring the long-term removal of CO₂ from the atmosphere since CO₂ is formed again in decomposition processes of biomass by aerobic processes. In addition to CO₂ emissions, long growth times of the biomass of from several years to decades must also be accepted when storing solid wood in the form of logs, for example.

Pyrolyzing biomass and depositing the resulting carbon, which is also referred to as biochar, or using it in agriculture, is also under discussion. This can also lead to CO₂ re-emissions, since carbon can in this case be exposed to the oxidizing effect of atmospheric oxygen. Furthermore, the energy contained in the biomass remains unutilized in this form. However, the biomass consists to a considerable extent of hydrogen, which would not produce CO₂ emissions if used for energy purposes. This potential is lost in the deposition or agricultural use of pyrolyzed biomass.

In addition, the application of pH-modifying chemicals such as mineral materials on areas of land or for ocean alkalinization may have as-yet unknown environmental impacts.

An object of the invention is that of separating CO₂ off from the atmosphere and/or from an offgas and reliably preventing re-emission. Furthermore, it is intended for the amount of energy required for this purpose to be reduced or at least some of the energy converted in the process to be utilizable.

### DISCLOSURE OF THE INVENTION

The invention proposes a process for removing and immobilizing carbon dioxide from the atmosphere and/or from an offgas, comprising the steps of:
a) converting the carbon dioxide from the atmosphere and/or from an offgas into biomass by means of photosynthesis, preferably on agricultural areas, in particular in a greenhouse,
b) conducting a biogas reaction in which the biomass produced in step a) is converted into biogas containing methane and carbon dioxide, in particular in a biogas reactor,
c) separating the methane off from the biogas obtained,
d) cracking the methane into carbon and hydrogen, with the carbon being obtained as a solid,
e) collecting the carbon obtained,
f) depositing the carbon obtained and
g) reacting carbon dioxide from the biogas with the hydrogen obtained in step d) to give a hydrocarbon mixture.

The process according to the invention comprises three biological/chemical reactions for removing CO₂ from the air. These are the photosynthesis in step a), the biogas reaction, in particular an anaerobic fermentation, in step b), and the cracking of the methane, in particular a pyrolysis of methane, in step d).

The photosynthesis takes place in step a). With photosynthesis, substances having a higher energy level are formed under the action of light from starting substances having a lower energy level, namely CO₂ and water. In nature, photosynthesis leads to the formation of biomass as well as products resulting therefrom such as biowaste and farm fertilizers, for example in the form of manure and slurry. The reaction of photosynthesis causes the growth of plants and algae in nature. Photosynthesis is also used for the industrial production of biomass in greenhouses or algae bioreactors.

Step a) for producing biomass can take place in an agricultural process, i.e. on an area used for agriculture, and/or in a greenhouse.

Biomass is defined in particular as substances and mixtures thereof that are produced by photosynthesis. These are also referred to as primary biomass. Biomass also refers to substances and mixtures thereof that have been formed as a result of the use of the primary biomass and have retained a biogenic character. In particular, the biomass may contain straw, forest residues, farm fertilizer, food residues and/or municipal waste.

In industrial applications, it is advantageous to optimize biomass production by adapting the reaction parameters such as the concentration of CO₂ in the ambient air. The efficiency of photosynthesis increases, depending on the plant, as the concentration of CO₂ increases. In C₄ plants such as corn, sugar cane or millet, maximum growth, i.e. maximum efficiency of photosynthesis, is already achieved under today's atmospheric conditions in terms of CO₂ concentration. In C₃ plants such as wheat, rye, barley, potatoes, soybeans and trees, the growth rates increase further with increasing CO₂ concentration. In step a), C₃ plants are preferably used for the conversion of CO₂ by means of biosynthesis. Maximum efficiency of photosynthesis in C₃ plants is achieved at more than 1000 ppm CO₂. Overall, by increasing the CO₂ concentration in the ambient air under optimal basic conditions in terms of temperature, irrigation and nutrient supply, in particular with nitrogen, up to 40% higher outputs can be achieved in biomass production, especially in greenhouses. The use of fast-growing C₃ plants can further increase the yield. An increased concentration of CO₂ also increases the growth rate of algae.

The offgas of which the CO₂ in step a) is possibly converted into biomass can for example be an offgas formed in the combustion of fossil fuels in a power plant. The offgas may for example also be a by-product which is formed in an industrial production process or the offgas can be an extraction gas which is produced in the extraction of fossil fuels such as coal, oil or natural gas. One example of an extraction gas is mine gas. The production of electric current from fossil fuels contributes the largest proportion of the offgases produced.

Industrial processes are similarly important as a source of climate-relevant offgases. One example of an industrial production process is cement production, in which the CO₂ emissions are mostly attributable to the calcination process. In the production of iron and steel and the auxiliary materials required for this purpose, the production offgases contain CO₂. Climate-relevant offgases can for example also arise in refineries.

These offgases, which are normally released into the atmosphere, can be collected after they have formed and be supplied to the proposed process.

Besides the carbon oxides already mentioned, industrial offgases usually contain other substances that have to be taken into account in the treatment of the offgas. These include inter alia methane, hydrogen, water vapor and the inert gas nitrogen, where the proportion of nitrogen can be up to 97% by volume. Further offgas constituents present can be impurities such as hydrogen sulfide, mercury and/or heavy metals. These impurities require pretreatment in order to achieve prescribed limits. Oxygen may also be found in concentrations of up to 6% by volume in the offgas from power plants. Besides methane, offgases formed during the extraction of, for example, natural gas contain CO₂ and nitrogen in relevant proportions.

Preferably, the offgas is cleaned prior to being supplied by conventional, already-known processes of dust, sulfur-containing compounds and other impurities such as nitrogen oxides, hydrogen chloride, hydrogen fluoride, mercury, other metals and other organic or inorganic substances.

The offgas supplied to step a) is in particular a mixture comprising CO₂. The offgas may additionally comprise, as further components, at least one inert gas such as nitrogen or argon and possibly water vapor.

Preferably, the offgas used in step a) originates at least in part from the use of the hydrogen produced in step d) or the biogas formed in step b), in particular the methane. The offgas in a pretreatment is optionally desulfurized, cleaned of the other impurities and/or dedusted. Further preferably, the offgas used in step a) consists exclusively of offgas which is recycled within the process. More preferably, the offgas used in step a) originates exclusively from the biogas produced in step b), in particular from the use of the hydrogen produced in step d). Accordingly, the offgas can originate from the biogas directly or indirectly, that is to say after the subsequent cracking of the methane.

The biogas reaction in step b) comprises in particular an anaerobic fermentation reaction. In the anaerobic fermentation the biomass is converted into biogas, that is to say a mixture of predominantly methane and carbon dioxide. The biogas produced in step b) preferably contains a total of at least 90% by volume, further preferably at least 95% by volume, of methane and CO₂, based on the overall biogas.

In particular, the biomass is hydrolyzed under the action of various microorganisms, such as bacteria. During the biogas reaction, there is preferably a pH in the range from 6 to 7, further preferably from 6.5 to 7 and in particular from 6.6 to 6.7. The temperature during the biogas reaction is preferably constant and is further preferably in a range from 25° C. to 45° C., more preferably from 30° C. to 40° C., in particular from 33° C. to 37° C., for example 35° C. In the biogas reaction, substances such as sugar, amino acids and/or fats are broken down. The hydrolysis is preferably followed by a fermentation or acetylation to form acids which are then converted into methane and carbon dioxide. The described reaction conditions represent an optimum for methanogenic bacteria.

As biomass, preference is given to using in step b) plants, in particular C₃ plants, biowaste, farm fertilizers such as manure and/or slurry, and/or municipal waste. The biomass produced in step a) can be used directly in step b). For instance, plants produced in step a) can be supplied directly to the biogas reaction in step b). Alternatively, the biomass produced in step a) can first be subjected at least in part to utilization or conversion, such that it is present in step b) in the form of biowaste, municipal waste or fertilizer.

The biogas reaction is preferably carried out in a closed vessel, which is also referred to as a fermenter. The biomass can be comminuted and optionally sorted prior to conducting the biogas reaction. The biomass can be supplied to the fermenter continuously. The residence time of the biomass in the fermenter is preferably more than one day. The biogas produced accumulates in particular in an upper region of the fermenter above a liquid and solid phase. The biogas preferably contains at least 40% by volume, further preferably 50% by volume to 75% by volume, in particular 62% by volume to 75% by volume, of methane, based on the total biogas. The biogas further contains CO₂ and possibly water vapor, hydrogen sulfide and/or ammonia.

The controlled fermentation, the biogas reaction, in particular in a closed vessel, offers the advantage that emissions, for example in the form of acids which may contaminate the groundwater or methane emissions which enter the atmosphere in an uncontrolled manner and thus contribute to climate change, are reduced.

The biogas produced contains CO₂, which can be separated in a workup of the biogas, in particular in a CO₂ separation apparatus, and supplied back to the photosynthesis. Step c) can accordingly also be referred to as the workup of the biogas. Polyimide hollow fiber membranes can for example be used to separate off the carbon dioxide.

In a step g), CO₂ from the biogas is reacted with hydrogen produced in step d) to give a hydrocarbon mixture.

Preferably, the CO₂ from the biogas after the separation of methane in step c) is supplied to the reaction with the hydrogen produced in step d). The hydrocarbon mixture obtained during the reaction of the CO₂ with hydrogen contains in particular kerosene, gasoline and/or waxes.

In order to convert the CO₂ from the obtained biogas into a hydrocarbon mixture, the CO₂can be supplied in particular to a Fischer-Tropsch apparatus.

As an alternative to a Fischer-Tropsch apparatus, the CO₂ can also be converted with the hydrogen produced in step d) to give methane and water. This can be done, for example, in a catalytic Sabatier reaction or in a biochemical process in which methanogenic microorganisms are used. Examples of suitable methanogenic microorganisms include Methanobacterium, Methanospirillium hungatii or Methanosaeta.

In addition to the reaction of CO₂ with the hydrogen obtained in step d) to give a hydrocarbon mixture, a portion of the CO₂ from the obtained biogas can be recycled into step a) after the separation of methane according to step c). In addition, a portion of the CO₂ from the obtained biogas can be liquefied after the separation of methane according to step c).

In order to increase the yield of solid carbon, it is further particularly preferable to introduce the hydrocarbons obtained in step g) at least in part into the reaction for cracking the methane in step d). In this case, not only the methane is cracked into carbon and hydrogen, but also the hydrocarbons present in the hydrocarbon mixture.

Preferably, the separation of the methane in step c) is effected physically, in particular by means of condensation, adsorption and/or a membrane process. The separation of the methane off from the biogas obtained in step c) is preferably conducted continuously.

For the separation of the methane, pressure swing adsorption, temperature-vacuum adsorption, chemical adsorption, membrane separation, compressed gas scrubbing processes and/or concentration swing adsorption are used in particular.

After the separation in step c), the material stream of the separated methane preferably contains more than 95% by volume of methane and more preferably not more than 1% by volume of CO₂.

In step d), which can also be referred to as pyrolysis, at least a portion of the methane formed in step b), and, if the hydrocarbons obtained in step g) are also recycled into step d), the hydrocarbons, are cracked into the elements hydrogen and carbon. The reactions that take place in step d) can be represented by the following reaction equations:

CH₄→2H₂+C

CH₃(CH₂)ₙCH₃→(n+3)H₂+(n+2)C

These reactions are endothermic, which means that energy must be supplied to the reaction system in order for the reaction to proceed.

Preferably, the cracking of the methane and optionally of the hydrocarbons in step d) is effected by means of a pyrolysis process and in particular at a temperature of at least 800° C., more preferably at least 1000° C., in particular more than 1200° C., more preferably more than 1400° C.

The energy required for conducting the above-mentioned reaction is preferably provided in the form of electricity from renewable sources such as wind, biogas and/or photovoltaics which in particular cannot be accepted at the time in the electricity grid. In particular, no fossil fuel is used for this purpose. Alternatively, a portion of the biogas or of the obtained hydrogen can be used to provide the energy needed for the cracking.

The cracking of the methane and optionally of the hydrocarbons can be conducted by means of thermopyrolysis, in which the energy needed is provided by direct or indirect heating. With regard to direct thermal cracking, processes are known in which methane and/or hydrocarbons flow in the form of bubbles through a column of liquid metal, are heated and cracked. Carbon accumulates on the surface of the metal column, hydrogen escapes and is supplied to further processing. Furthermore, a thermal process is known in which the cracking is performed in a moving bed consisting of carbon granules.

In addition, the cracking of the methane and optionally of the hydrocarbons can be conducted using an arc, with the energy being supplied by electric discharge in the gas phase. Such a process is known to those skilled in the art as the Kværner process. The cracking of the methane and optionally of the hydrocarbons can accordingly be conducted in a plasma hydrogen generator. The plasma is produced, for example, by irradiation with electromagnetic waves. The plasma cracking of the methane and optionally of the hydrocarbons takes place at temperatures of up to 2000° C. and at locally high energy density.

The cracking of the methane and optionally of the hydrocarbons can alternatively be performed in the presence of at least one catalyst at elevated temperature, in particular in a range from 800° C. to 1000° C. The catalyst used can be at least one metal, in particular selected from the group consisting of Fe, Ni and Cr, and/or activated carbon.

The pyrolysis produces a gas phase, containing hydrogen as a gaseous product, and carbon, in particular elemental carbon, as a solid. This reaction cracks the energy carrier methane and optionally the hydrocarbons into two substances. The hydrogen can be used for energy purposes without concerns in terms of impact on the climate, since the combustion of hydrogen with air does not produce a greenhouse gas, but rather water or water vapor. If, as an alternative, the combustion of soot was conducted in order to generate the corresponding energy, 3.67 kg of climate-damaging CO₂ would for example be produced by the combustion of 1 kg of soot.

The gas phase after the pyrolysis preferably comprises hydrogen, an inert gas, methane and possibly hydrocarbons. The gas phase can be supplied at least in part to a separation apparatus, in particular a membrane, to produce hydrogen and a residual gas.

A filter in which the carbon is separated from the hydrogen is preferably connected downstream of step d). The conversion of the methane and optionally the hydrocarbons to hydrogen and carbon is preferably effected with a yield of approx. 96% to 97%.

Preferably, after the cracking of the methane and optionally of the hydrocarbons in step d), the hydrogen is separated off from the residual gas, that is to say further gaseous components, in particular by means of a membrane process.

The hydrogen can be used as material or for energy purposes and to this end can be supplied to a power plant or a transport and distribution system. The residual gas remaining after the separation of the hydrogen off from the gas phase is preferably supplied to the power plant. The power plant preferably comprises a gas engine for generating electricity and heat. The heat generated in the power plant can at least in part be used to generate cold. The gas phase can also already, that is to say without the separation of the hydrogen, be supplied to the power plant.

The electricity generated in the power plant is preferably used for the operation of the reactor for cracking methane and optionally the hydrocarbons and/or fed into the public power grid in order to stabilize the grid. The power plant also produces heat and offgas, which can be used to produce the biomass in step a), in particular in a greenhouse.

In a preferred embodiment, a portion of the hydrogen is recycled to the reactor for cracking the methane and optionally the hydrocarbons.

Preferably, at least a portion of the hydrogen produced in the cracking is used as a starting material in the chemical industry, as an energy carrier for the generation of electric current and/or heat, where the heat can optionally be used at least in part for the generation of cold, or as a fuel for vehicles. For the generation of energy, the hydrogen can be mixed with combustion air and combusted in a gas turbine. The gas turbine can drive a power generator that generates electric current. The offgases from the combustion can be used to generate process steam via a steam generator.

It is also conceivable to convert the hydrogen into electric current via a fuel cell or to use the hydrogen for other purposes, for example for refueling hydrogen-powered vehicles or for heating.

Preferably, at least a portion of the hydrogen produced in step d)/of the biogas produced in step b) will be used as an energy source for cracking the methane and optionally the hydrocarbons in step d). The energy required for the cracking according to step d) is preferably provided at least in part by using the hydrogen produced in step d) and optionally the residual gas and/or is generated from renewable sources. Alternatively or in addition, the methane separated off in step c) can also be used to generate electricity and/or heat. For example, 30% to 50% by volume of the biogas produced, in particular in the form of methane and/or hydrogen, can be used to generate electricity and/or heat.

The hydrogen produced in step d) is preferably used at least in part as a starting material for syntheses in the chemical industry, as an energy carrier for the generation of electric current, heat, optionally cold, and/or as a fuel for vehicles.

The hydrogen is preferably separated off from the carbon after the cracking of the methane and optionally of the hydrocarbons according to step d), in particular by means of filtration. The hydrogen separated off is supplied to step g) together with the CO₂ from the biogas in order to produce hydrocarbons, in particular methane. This can considerably increase the yield of solid carbon.

The carbon obtained as a solid in step d) is in particular not used as an energy carrier. The carbon is preferably collected, transported to a deposition site and subjected to long-term storage or final storage. The carbon obtained in step d) is preferably completely deposited. The carbon obtained in step d) is further preferably separated off from the gas phase containing the hydrogen.

The carbon obtained in step d) is further preferably mixed with other components, in particular further solids, prior to deposition in step f) such that use for energy purposes is no longer possible, in order to ultimately immobilize the carbon and be able to store it particularly securely. The further solids used can include for example sand, loam, gravel, construction rubble, slags, stones, waste, in particular from industrial dismantling, or a combination of two or more of these materials. Accordingly, the carbon obtained in step d) is preferably immobilized, and by mixing the obtained carbon with a further solid a long-term immobilization is ensured. The solids mixture formed, which comprises the obtained carbon with at least one further solid, is stored in particular geologically and for the long term in a carbon sink, such as a mine.

The carbon obtained as a solid in step d) is preferably mixed with at least one further solid, in particular sand and/or rock, before being deposited according to step f).

The deposition in step f) preferably extends to at least 30 years, further preferably at least 50 years.

The carbon is preferably deposited underground, for example in old mines, in particular potash mines or salt mines. However, the carbon is also suitable as a filling material for filling open pit mines, quarries, gravel, gypsum or clay pits. In order to avoid geological damage and to comply with nature conservation obligations, decommissioned mining sites are subjected to extensive rehabilitation and recultivation measures. This involves filling the cavities of the subsurface mines as well as the pits themselves with mineral material. Suitable materials used are construction rubble, slags, stones, waste from industrial dismantling, and other industrial waste having sufficient strength. If carbon is used alone, it would have to first be compressed for the filling. Mixing the pulverulent carbon with minerals or waste proves to be advantageous, since carbon can penetrate into the porous structure of the minerals and be lastingly fixed there.

The deposition in step f) of the process according to the invention can also be understood to mean the use of the carbon formed as a solid in the construction industry, in particular in road construction, and/or in agriculture.

The material use of solid-formed carbon in industrial products such as sheaths of electrical cables, insulation materials, insulators, underground structures, etc. can also be classified as deposition as long as it is ensured that the lifetime of the applications extends to at least 30 years, more preferably at least 50 years. In this case, the carbon is mixed generally in solid form into the material used for the products.

Preferably, deposition comprises exclusively geological, especially underground, deposition, for example in mines.

At least steps a) to e) and step g) are preferably performed in one module. A module is understood to mean a spatially cohesive unit. Hydrocarbons are further preferably produced in the module, using CO₂ and the residual gas.

Furthermore, two or more modules are preferably installed in different locations.

Preferably, in at least two modules, that is to say in at least two respectively spatially cohesive units, in each case at least steps a) to e) and g) are conducted and further preferably the at least two modules are connected to a central control station.

Preferably, the mixing of the obtained carbon with a further solid is conducted outside or independently of the modules. The obtained carbon originates from at least one module. The mixing can be connected, in particular in terms of data technology, to the central control station.

### ADVANTAGES OF THE INVENTION

By means of the described process, the CO₂ “extracted” from the atmosphere and/or offgas is removed from the atmosphere. A lasting immobilization of the carbon is possible and thus a cleaning effect for the atmosphere is achieved.

Compared to combustion of this carbon, geological storage and corresponding binding of 3.67 kg of CO₂ equivalent can thus be achieved with deposition of 1 kg of carbon.

The combination of photosynthesis, anaerobic fermentation, pyrolysis and deposition, that is to say steps a), b), d) and f), thus enables the reduction of CO₂ in the atmosphere. In contrast to the above-described known other processes for the separation of CO₂ off from the atmosphere, the process according to the invention forms hydrogen and possibly biogenic CO₂, which can be used economically.

The conversion of CO₂ into solid-form carbon considerably simplifies sequestration, since a solid is immobilized and not gas. Finding a suitable carbon deposit appears to be much easier than finding suitable storage capacities for gaseous CO₂, as are required for conventional CCS processes.

In addition, the deposition of carbon in solid form eliminates the risks of re-emission. The geological risks that accompany the injection of gaseous carbon dioxide under high pressure are absent.

The reaction of the CO₂ contained in the biogas with the hydrogen obtained in the cracking to give hydrocarbons including methane has a positive effect on the yield of solid carbon. The amount of carbon can be increased by up to twofold as compared to a process without introduction of the hydrocarbons into the cracking. This consequently amplifies the desired effect of cleaning the atmosphere of climate-damaging CO₂.

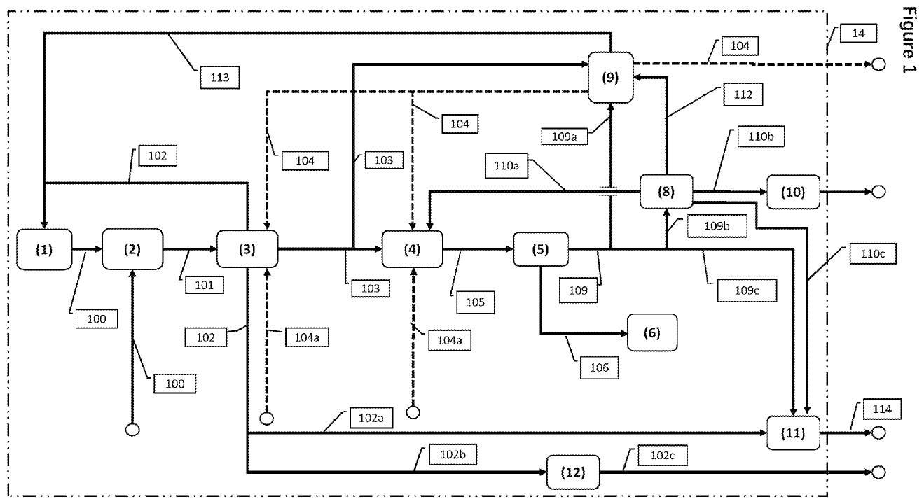

The invention is described in more detail with reference to the following drawings (FIGS. 1-4), the list of reference signs and the claims, and also the examples.

In the drawings:

FIG. 1 shows a diagram of the process according to the invention in a first embodiment,

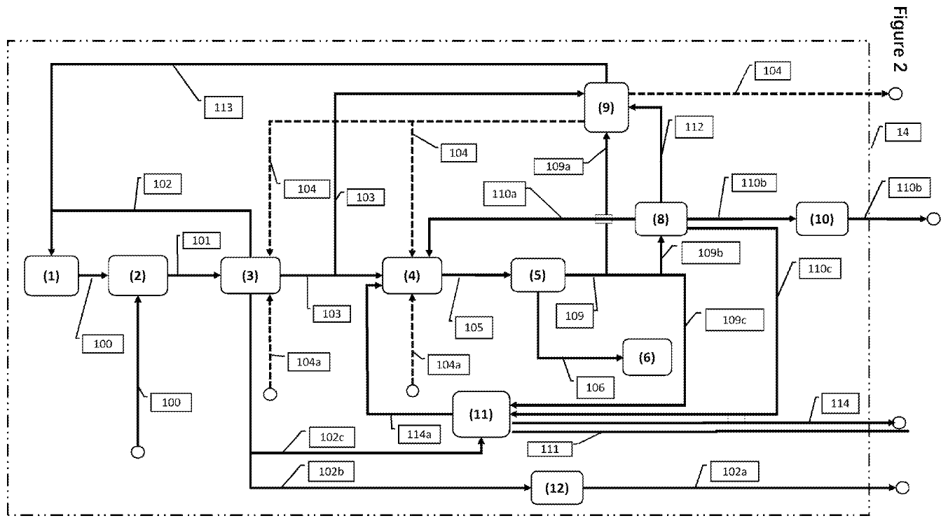

FIG. 2 shows a diagram of the process according to the invention in a second embodiment,

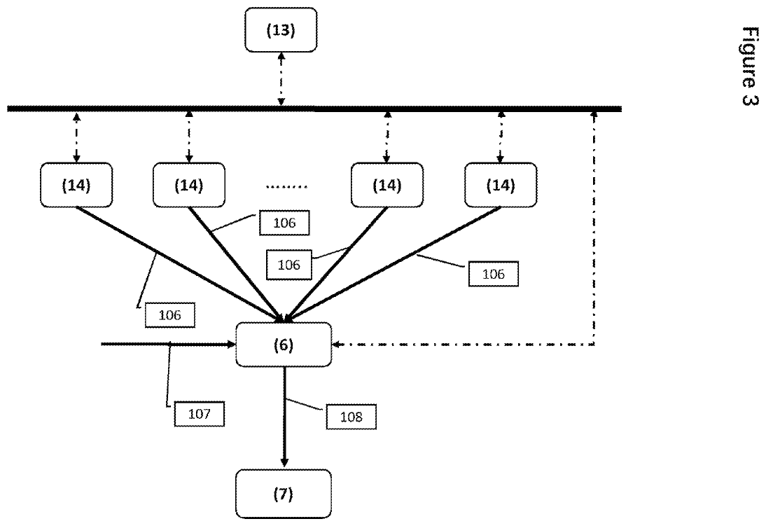

FIG. 3 shows a diagram of a connection of a plurality of modules for conducting the process according to the invention and

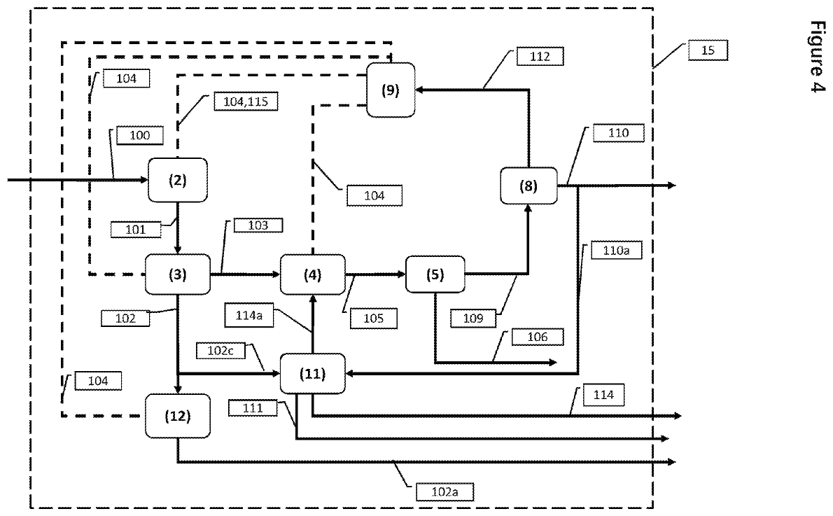

FIG. 4 shows a section of a module for conducting a plurality of steps of the process according to the invention,

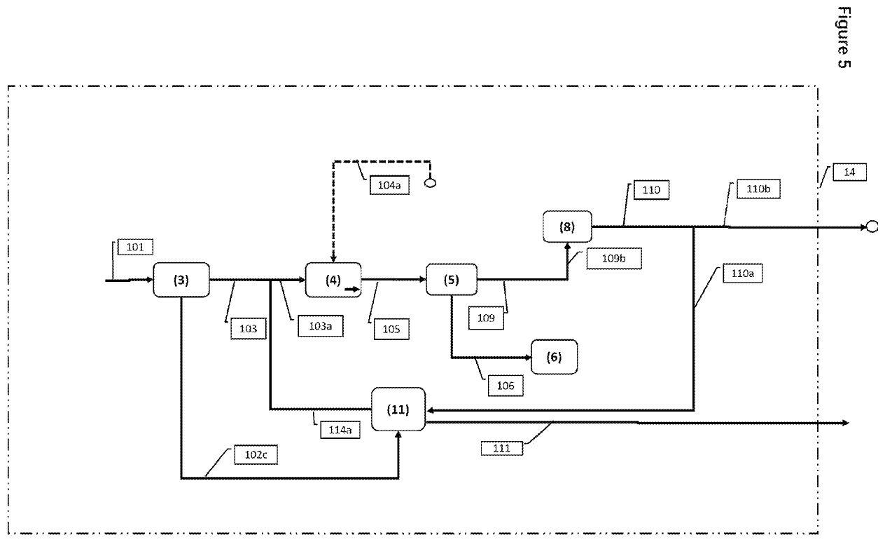

FIG. 5 shows a process diagram of the first process conducted in example 4,

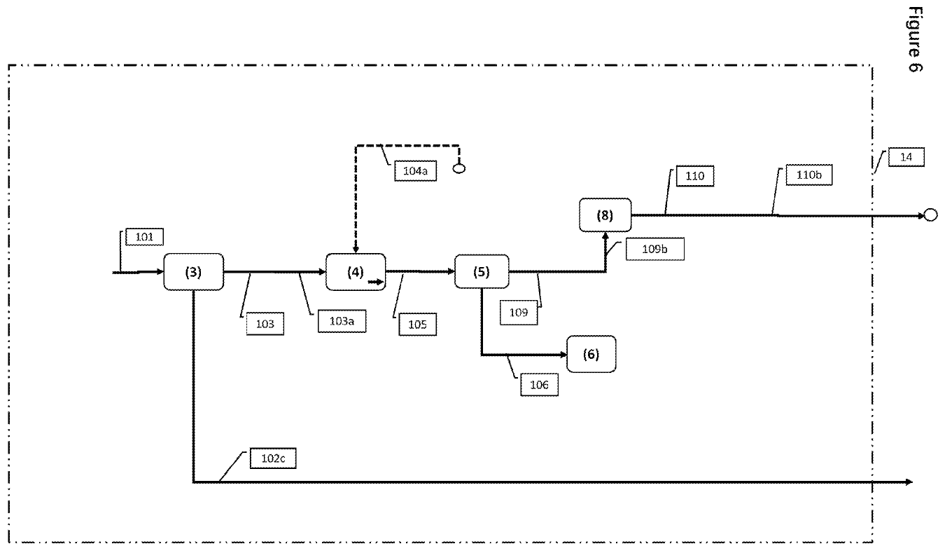

FIG. 6 shows a process diagram of the second process conducted in example 4.

FIG. 1 shows a diagram of the process according to the invention, where the solid arrows indicate material flows and the dashed arrows indicate energy flows. The circles represent connecting points with the surroundings of the system and the dash-dotted line marks the boundaries of the module 14 under consideration.

In step a), biomass 100 is produced by photosynthesis from CO₂ 102. This is done, for example, by growing plants in a field or, alternatively or additionally, by cultivating plants in a greenhouse 1. By means of targeted selection of the plants, rapid growth of the biomass 100 can be promoted and the suitability for the production of biogas 101 can be optimized.

To increase plant growth, the air in the greenhouse 1 can be enriched with CO₂ 102 and heated. The CO₂ 102 can be supplied separately and/or as part of an offgas 113 to the greenhouse 1. The CO₂ 102 is preferably recycled from further steps of the process, in particular from a workup 3 of biogas 101 produced in the process and as part of the offgas 113 from a combined heat and power (CHP) plant 9.

The biomass 100 is fermented in step b) of the process, in particular with an anaerobic bacterial reaction, to give biogas 101, which contains methane 103 and CO₂ 102. The reaction is conducted in particular continuously in a mixing vessel constituting a biogas reactor 2.

In step c) of the process, the biogas 101 is supplied to a workup 3, wherein methane 103 in the form of a methane-rich stream is separated off from a stream containing CO₂ 102. The CO₂ 102, for example the entire stream containing CO₂ 102, can be recycled into the greenhouse 1 in order to enrich the air in the greenhouse 1 with CO₂ 102. Alternatively or in addition to the recycling of the CO₂ 102 from the workup 3 into the greenhouse 1, at least a portion 102a of the CO₂ 102 is conducted to a Fischer-Tropsch and/or methanation apparatus 11.

A further portion 102b of the CO₂ can be supplied to a CO₂ liquefaction 12, from which liquid CO₂ 102c can then be withdrawn.

In step d) of the process, the methane 103 from the workup 3 is supplied to a reactor 4. It is subjected to intense heating there. In the reactor 4, methane 103 is cracked into its constituents carbon as a solid 106 and hydrogen 110. All reaction types mentioned, that is to say the reaction by means of pyrolysis, for example thermopyrolysis, electric arc pyrolysis, plasma pyrolysis or catalytic pyrolysis, can be used here.

The conversion of the methane 103 is not complete and other hydrocarbons are also produced in small quantities. At the outlet of the reactor 4, a product gas mixture 105 is formed, which contains gaseous methane 103, hydrogen 110 and other hydrocarbons 114 and also carbon as a solid 106.

For the operation of the reactor 4, preference is given to using energy such as electric current 104a from renewable sources, in particular from wind energy and photovoltaics. The electric current 104 can alternatively or additionally be generated in the CHP plant 9.

Connected downstream of the reactor 4 is a filter 5 in which the separation of the solid 106 from a gas phase 109, which contains hydrogen 110, takes place.

At least a portion 109a of the gas phase 109 may optionally be intermediately stored and supplied as fuel to the integrated CHP plant 9. Furthermore, the electric current 104 generated in the CHP plant can be fed into the public power grid in order to stabilize the grid.

Alternatively, the hydrogen 110 can be separated in an H₂ separation apparatus 8, for example a pressure swing adsorption system or by means of membranes, from residual gas 112 present in the gas phase 109. To this end, at least a portion 109b of the gas phase 109 is supplied to the membrane 8. A portion of the hydrogen 110a can be recycled into the reactor 4 and a further portion can be used further as product 110b. To this end, the hydrogen used further as product 110b can first be supplied to a device 10 for preparation of the H₂ for onward conveyance and, for example, compressed and filled into gas cylinders or introduced into a pipeline for further transport.

According to the invention, at least a portion 109c of the gas phase 109 or alternatively of the hydrogen 110c is supplied to the Fischer-Tropsch and/or methanation apparatus 11. Here, the hydrogen from the gas phase 109 or the hydrogen 110 together with CO₂ 102 originating from the workup 3 can be converted into a hydrocarbon mixture 114. The hydrocarbon mixture 114 preferably contains kerosene, gasoline, waxes and mixtures thereof.

As an alternative to the Fischer-Tropsch apparatus 11 mentioned here, it is also possible to use any other process known to those skilled in the art for the production of hydrocarbons, for example a catalytic Sabatier reaction or a biochemical process in which methanogenic microorganisms are used. In the Fischer-Tropsch and/or methanation apparatus 11 or in the process alternatively used, longer-chain hydrocarbons, for example kerosene, gasoline and/or waxes, can be produced, or also methane or shorter-chain hydrocarbons such as ethane, propane or butane, and also mixtures thereof.

The residual gas 112 used after the separation from the gas phase 109 and serving as fuel can optionally be intermediately stored and optionally supplied to the CHP plant 9. As an alternative to an external power source, the electric current 104 generated in the CHP plant 9 can be used for the cracking of the methane 103 in the reactor 4. Furthermore, the offgases 113 produced in the CHP plant 9 are preferably supplied, in particular together with the heat 115 generated therein, to the greenhouse 1.

The process illustrated in FIG. 1 can be combined locally to form a module 14. A module 14 can serve for the local supply of electricity 104, hydrogen 110 and heat 115 and also for the production of hydrocarbons 114.

FIG. 2 shows an alternative embodiment of the process according to the invention, which differs from the embodiment shown in FIG. 1 in particular by the introduction of the hydrocarbons and/or methane produced in the Fischer-Tropsch and/or methanation apparatus 11 into the reactor 4.

By introducing at least a portion 114a of the hydrocarbons and/or methane produced in the Fischer-Tropsch and/or methanation apparatus into the reactor 4, the yield of solid carbon 106 can be markedly increased.

In this case, in the reactor not only is the methane 103 converted into carbon 106 and hydrogen 110, but also the hydrocarbons and/or methane 114a introduced into the reactor and which were obtained in the Fischer-Tropsch reaction are cracked into solid carbon 106 and hydrogen 110. The reaction conducted is the same as described above for FIG. 1 for the cracking of the methane.

For the conversion of the CO₂ to hydrocarbons 114, 114a in the Fischer-Tropsch and/or methanation apparatus, in addition to the portion 109c of the hydrogen-containing gas phase 109, a portion 110a of the hydrogen separated off in the H₂ separation apparatus 8 is also introduced into the Fischer-Tropsch and/or methanation apparatus 11. As an alternative to the embodiment illustrated here, it is also possible to introduce only the portion 109c of the hydrogen-containing gas phase 109 or only the hydrogen 110a into the Fischer-Tropsch and/or methanation apparatus 11.

It is also possible, as illustrated here, to withdraw a portion of the hydrocarbons and/or methane 114 obtained in the Fischer-Tropsch and/or methanation apparatus 11 as a product and to introduce only a portion 114a into reactor 4. However, in order to obtain the maximum yield of solid carbon 106, it is preferable to introduce all of the hydrocarbons produced in the Fischer-Tropsch and/or methanation apparatus 11 and/or all of the methane produced into the reactor 4.

FIG. 3 shows a diagram of a connection of a plurality of modules 14, where the solid arrows indicate material flows and the dashed-line arrows indicate the data flow. The modules 14 have an internal, that is to say local, transport and distribution system for the distribution of the hydrogen 110.

The modules 14 are connected to each other in terms of data technology, so that an optimized regime is possible with respect for example to the product portfolio, load or distribution. The control of the modules 14 is optimized from a control station 13 at which data is centrally recorded, stored and processed. The control station 13 and the modules 14 communicate with each other via a data network such as the internet.

The carbon arising in the modules 14 as a solid 106 is processed at a central mixing apparatus 6, which is likewise connected to the control station 13, with further solids 107, which are also referred to as fillers, such as sand or bulk material, to give a solids mixture 108. The solids mixture 108 can now be geologically and lastingly stored in a carbon sink 7 such as for example an old pit. The carbon as a solid 106 can also be used in other applications in which it is lastingly immobilized, so that it can be considered equivalent to the CO₂ removed from the air. Alternatively, the carbon can also be mixed into materials that are used to produce sheaths for electrical cables, insulators, insulation materials or to seal underground structures.

FIG. 4 shows a section of a module 14 in the air-cleaning mode. The balance boundary 15 corresponds to example 3 explained below. Biomass 100 is supplied to the biogas reactor 2, which also receives electric current 104 and heat 115 from the CHP plant 9. The biogas 101 withdrawn from the biogas reactor 2 is separated in the workup 3 into (bio) methane 103 and gaseous CO₂ 102. The methane 103 is in turn converted in the reactor 4 into a product mixture 105, that is to say a carbon-gas mixture, with carbon being discharged in the filter 5 as solid 106, for example as soot.

At least a portion 102a of the gaseous CO₂ is supplied to a Fischer-Tropsch and/or methanation apparatus 11 for the production of hydrocarbons and/or methane. A portion 114a of the hydrocarbons is supplied to the reactor 4 for cracking into solid carbon 106 and hydrogen 110. After separation from the residual gas obtained in the reactor, at least a portion 110a of the hydrogen is supplied to the Fischer-Tropsch and/or methanation apparatus 11.

If not all of the hydrocarbons 114a produced in the Fischer-Tropsch and/or methanation apparatus 11 are introduced into the reactor 4, a portion of the hydrocarbons 114 can be withdrawn as product.

If not all of the CO₂ is supplied to the Fischer-Tropsch and/or methanation apparatus 11, the remaining CO₂ can be supplied to a CO₂ liquefaction 12 and be withdrawn from the latter as liquid CO₂ 102c.

Water 111 formed in the Fischer-Tropsch and/or methanation reaction is condensed and removed from the apparatus.

The gas phase 109 separated off in the filter 5 is conveyed via the H₂ separation apparatus 8 at which hydrogen 110 is available. The remaining residual gas 112 is used for energy purposes in the CHP plant 9, with electric current 104 being used within the module 14 for liquefying the CO₂ 102 and electric current 104 and heat 115 being used in the biogas reactor 2 and also electric current 104 being used for cracking the methane 103 in the reactor 4.

### EXAMPLES

### Exemplary embodiment 1: Removal of CO₂ from the Air with Cultivation of Corn

For the quantification of the CO₂ emissions, a subsystem of a module is analyzed, comprising a reactor, a filter and an H₂ separation apparatus for hydrogen separation and also a CHP plant. It is further assumed that no external energy, that is to say from outside the system, is supplied, which corresponds to a setting of the currents 104a in FIG. 1 equal to 0.

Depending on the composition of the biomass used, the methane mixture, that is to say the biogas produced, has already nominally been associated to different degrees with CO₂ emissions from the upstream process steps, in particular of the production of the biomass. In this exemplary embodiment, corn is considered as the biomass used. The following emissions from the production of the corn can already be attributed to the methane mixture formed from corn:

TABLE 1

| Operation | Equivalent CO₂ emissions | |
|---|---|---|
| Cultivation | 17.7 | g CO₂ eq./MJ |
| Processing (fermentation in step b)) | 4.3 | g CO₂ eq./MJ |
| Workup of the biogas | 4.5 | g CO₂ eq./MJ |
| Total | 26.5 | g CO₂ eq./MJ |

The subsystem analyzed is operated such that 60% by weight of the methane produced from the com is converted stoichiometrically into hydrogen and carbon. The remaining 40% by weight of the methane is used for the production of electric current, which in FIG. 1 is taken from the CHP plant 9 with the reference 104.

The following calculation is based on a consumption of 100 kg of methane.

The CHP plant generates electricity with an efficiency of 36%. CO₂ emissions are attributed to the generated electricity as follows:

$$40 kg of {CH}_{4} \times 36 \% \times 50 MJ / kg \times 26.5 g of {CO}_{2} eq . / MJ = 19.08 kg of {CO}_{2} eq .$$

The two products carbon as a solid and hydrogen are formed in the cracking of the methane. When using 60 kg of methane, to which equivalent CO₂ emissions are attributed as follows, 45 kg of carbon and 15 kg of hydrogen are formed:

$$60 kg \times 50 MJ / kg \times 26.5 g of {CO}_{2} eq . / MJ = 79.5 kg of {CO}_{2} eq .$$

The formed carbon as a solid is lastingly bound since it is deposited. This solid carbon is of biogenic origin, so a credit for non-emissions is taken into consideration, since these emissions, which for example would have occurred during the burning of the solid carbon, are not delivered to the atmosphere. 1 kg of bound solid carbon corresponds to a credit of 3.67 kg of CO₂ equivalent (eq.). For 45 kg of solid carbon to be deposited here, a credit of 165.15 kg of CO₂ eq. thus results.

The 15 kg of hydrogen produced, which considering the calorific value of hydrogen of 120 MJ/kg of H₂ corresponds to 1800 MJ of usable energy (15 kg×120 MJ/kg of H₂=1800 MJ), is assigned 79.5 kg of CO₂ eq. (44.1 g of CO₂ eq./MJ of H₂) from electricity generation in the CHP plant and is attributed approx. 1 g of CO₂ eq./MJ from the transport and handling of the solid carbon during long-term storage. The greenhouse gas quota for hydrogen is then 54.1 g of CO₂ eq./MJ of H₂.

The credit from the storage of the solid carbon is as stated above −165.15 kg of CO₂ eq. per 100 kg of methane (−91.75 g of CO₂ eq./MJ of H₂). If this credit is taken into consideration in the accounting of the hydrogen produced, an advantageous CO₂ balance of the hydrogen produced of-46.65 g of CO₂ eq./MJ of H₂ is demonstrated (−46.65=44.1 +1.00−91.75).

### Exemplary Embodiment 2: Removal of CO₂ from the Atmosphere Using Slurry

If slurry is used instead of corn to produce the methane in the biogas reactor, the hydrogen, without taking into consideration the credit for the solid carbon stored, is attributed a CO₂ emission of −212.48 g of CO₂ eq./MJ of H₂ and, taking into consideration the credit for depositing the solid carbon, is attributed −304.23 g of CO₂ eq./MJ of H₂.

### Example 3: Comparison of the Process According to the Invention with Known DAC Processes

The following conditions are specified for the comparison of the process according to the invention, which can also be referred to as an air cleaning process, with the DAC (direct air capture) process.

The removal of 1 t of CO₂ from the ambient air is considered for both processes.

Based on S. Deutz, A. Bardow “Supplementary Material: Life-cycle assessment for industrial direct air capture process based on temperature-vacuum swing adsorption”, Nature Energy, 6, pages 203 to 213 (2021), it is assumed that approx. 0.7 kWh per kg of CO₂ removed from the air is required for mechanical work such as the operation of fans and pumps and an additional 3.3 kWh of heat are required.

When heat pumps are used, 1.3 kWh of electrical power is required as a heat substitute, resulting in a total energy requirement including liquefaction of 4.11 or 2.11 kWh/kg CO₂.

With regard to the air cleaning method according to the invention and illustrated correspondingly in FIG. 3, the energy balance takes into account the production of biogas from biomass in the biogas reactor 2, the separation of the biogas by means of membranes in the workup 3 into CO₂ and methane, the cracking of methane by means of electricity generated within the process in the reactor 4, with the electricity having been generated by means of the CHP plant 9, the separation of the solid carbon in the filter 5, which has been recorded by means of CO₂ equivalents, the separation of the hydrogen via the H₂ separation apparatus 8 and the liquefaction of CO₂ in the CO₂ liquefaction plant 12.

The removal of 1 t of CO₂ from the air with the aid of the DAC process according to the prior art requires 2.11 MWh of electrical power, while the removal by means of the air cleaning process removes 1 t of CO₂ in the form of 157 kg of carbon and 423 kg of CO₂ as a gas, where the energy required for the described process steps is obtained exclusively from biogas and additionally 5 kg of hydrogen and 1.1 MWh of heat are produced, with temperatures of up to 500° C. being available.

### Example 4: Increase in the Carbon Yield by Means of CO₂ Methanation

The system used in this example comprises a biogas workup 3, a cracking reactor 4, a carbon filter 5, an H₂ separation apparatus 8 and a methanation reactor 11. In contrast to the above examples, the energy source used is electricity from renewable sources (renewable electricity). For the purposes of simplification, it is also assumed that renewable electricity is not associated with any greenhouse gas pollution. The proportion of methane in the biogas is 58% by volume. The yields of the reactions correspond to the stoichiometry. The process diagram is illustrated in FIG. 5.

First, the biogas is separated into methane 103 and CO₂ 102c in the biogas workup 3. The carbon dioxide 102c is supplied to the methanation reactor 11, where it is reacted with hydrogen 110a, obtained in the H₂ separation apparatus 8, to give methane 114a. The methane 114a produced in the methanation reactor 11 is supplied together with the methane 103 from the apparatus 3 to the cracking reactor 4, where it is converted into solid carbon 106 and gaseous hydrogen 110. Carbon is separated off in filter 5, hydrogen is purified in apparatus 8 and divided into two streams. A first stream 110a is recycled to the methanation reactor and a second stream 110b is compressed and sold as a product.

The mass balance of the process is shown in table 2.

Alternatively, the conversion in the methanation process of the CO₂ contained in the biogas can be dispensed with. This is illustrated as an example in FIG. 6. In this case, CO₂ is emitted as a gas into the atmosphere or is liquefied and sequestered. The mass balance of the process is shown in table 3.

The partial recycling of the hydrogen in the process according to FIG. 5 increases the yield of the solid carbon by about 70%. The yield of hydrogen due to the methanation reaction is reduced accordingly.

TABLE 2

| Stream | Mass flow | CH₄ | CO₂ | H₂ | C | H₂O |
|---|---|---|---|---|---|---|
| no. | [kg/h] | [kg/h] | [kg/h] | [kg/h] | [kg/h] | [kg/h] |
| 101 | 332 | 112 | 220 | — | — | — |
| 102c | 220 | — | 220 | — | — | — |
| 103 | 112 | 112 | — | — | — | — |
| 114a | 80 | 80 | — | — | — | — |
| 103a | 192 | 192 | — | — | — | — |
| 105 | 192 | — | — | 48 | 144 | — |
| 106 | 144 | — | — | — | 144 | — |
| 109 | 48 | — | — | 48 | — | — |
| 110 | 48 | — | — | 48 | — | — |
| 110a | 40 | — | — | 40 | — | — |
| 110b | 8 | — | — | 8 | — | — |
| 111 | 180 | — | — | — | — | 180 |

TABLE 3

| Stream | Mass flow | CH₄ | CO₂ | H₂ | C | H₂O |
|---|---|---|---|---|---|---|
| no. | [kg/h] | [kg/h] | [kg/h] | [kg/h] | [kg/h] | [kg/h] |
| 101 | 332 | 112 | 220 | — | — | — |
| 102c | 220 | — | 220 | — | — | — |
| 103 | 112 | 112 | — | — | — | — |
| 114a | — | — | — | — | — | — |
| 103a | 112 | 112 | — | — | — | — |
| 105 | 112 | — | — | 28 | 84 | — |
| 106 | 84 | — | — | — | 84 | — |
| 109 | 28 | — | — | 28 | — | — |
| 110 | 28 | — | — | 28 | — | — |
| 110a | — | — | — | — | — | — |
| 110b | 28 | — | — | 28 | — | — |
| 111 | — | — | — | — | — | — |

### LIST OF REFERENCE NUMERALS

1 Greenhouse
2 Biogas reactor
3 Workup of biogas
4 Reactor
5 Filter
6 Mixing apparatus
7 Carbon sink
8 H₂ separation apparatus
9 Combined heat and power plant
10 Device for preparation of the H₂ for onward conveyance
11 Fischer-Tropsch and/or methanation apparatus
12 CO₂ liquefaction
13 Control station
14 Module
15 Balance boundary
100 Biomass
101 Biogas
102, 102a, 102b CO₂
102c Liquid CO₂
103 Methane
103a Methane-hydrocarbon mixture
104 Electric current
104a External energy
105 Product mixture
106 Carbon as a solid
107 Further solid
108 Solids mixture
109, 109a, 109b, 109c Gas phase
110, 110a, 110b, 110c Hydrogen
111 Water
112 Residual gas
113 Offgas
114, 114a, 114b Hydrocarbon mixture
115 Heat

1-19. (canceled)

20. A process for removing and immobilizing carbon dioxide from the atmosphere and/or from an off-gas, comprising the steps of:
a) converting the carbon dioxide from the atmosphere and/or from an off-gas into biomass by means of photosynthesis,
b) conducting a biogas reaction in which the biomass produced in step a) is converted into biogas containing methane and carbon dioxide,
c) separating the methane off from the biogas obtained,
d) cracking the methane into carbon and hydrogen, with the carbon being obtained as a solid,
e) collecting the carbon obtained,
f) depositing the carbon obtained and
g) reacting carbon dioxide from the biogas with the hydrogen obtained in step d) to give a hydrocarbon mixture.

21. The process according to claim 20, wherein the carbon dioxide is converted into biomass by means of photosynthesis on agricultural areas.

22. The process according to claim 20, wherein the carbon dioxide from the obtained biogas is recycled into step a) after the separation of methane according to step c).

23. The process according to claim 20, wherein at least a portion of the hydrocarbon mixture obtained in step g) is introduced into step d) and cracked into carbon and hydrogen.

24. The process according to claim 20, wherein the biogas produced in step b) contains at least 40% by volume of methane.

25. The process according to claim 20, wherein the biogas reaction comprises an anaerobic fermentation reaction.

26. The process according to claim 20, wherein the separation of the methane in step c) is effected by means of condensation, adsorption and/or a membrane process.

27. The process according to claim 20, wherein the cracking of the methane in step d) is effected by means of a pyrolysis process.

28. The process according to claim 20, wherein the hydrogen after the cracking of the methane according to step d) is separated off from the carbon.

29. The process according to claim 28, wherein the hydrogen after the cracking of the methane according to step d) is separated off from the carbon by means of filtration.

30. The process according to claim 20, wherein after the cracking of the methane in step d), the hydrogen is separated off from a residual gas.

31. The process according to claim 30, wherein the hydrogen is separated off from the residual gas by means of a membrane process.

32. The process according to claim 20, wherein the hydrogen produced in step d) is used at least in part as a starting material for syntheses in the chemical industry, as an energy carrier for the generation of electric current and/or heat, and/or as a fuel for vehicles.

33. The process according to claim 32, wherein the energy required for the cracking according to step d) is provided at least in part by using the hydrogen produced in step d) and optionally the residual gas and/or is generated from renewable sources.

34. The process according to claim 32, wherein the off-gas used in step a) originates at least in part from the use of the hydrogen produced in step d) and in a pretreatment is optionally desulfurized, cleaned of other impurities and/or dedusted.

35. The process according to claim 20, wherein at least steps a) to e) and optionally step g) are carried out in a module.

36. The process according to claim 35, wherein at least steps a) to e) and optionally step g) are each carried out in at least two modules, and the at least two modules are connected to a central control station.

37. The process according to claim 20, wherein the carbon obtained as a solid in step d) is mixed with at least one further solid before being deposited according to step f).

38. The process according to claim 37, wherein the at least one further solid is sand and/or rock.

39. The process according to claim 20, wherein step b) is conducted in a biogas reactor.
